(19) United States
(12) Patent Application Publication
(10) Pub. No.: US 2020/0138335 A1
(43) Pub. Date: May 7, 2020

(54) DEVICE AND PROCESS FOR ELECTRICAL IMPEDANCE TOMOGRAPHY (EIT) WITH IDENTIFICATION OF A HEART REGION

(71) Applicant: Drägerwerk AG & Co. KGaA, Lübeck (DE)

(72) Inventor: Birgit STENDER, Lübeck (DE)

(21) Appl. No.: 16/670,047

(22) Filed: Oct. 31, 2019

(30) Foreign Application Priority Data
Nov. 1, 2018 (DE) 102018008545.8

(51) Int. Cl. A61B 5/08; A61B 5/053; A61B 5/00

(52) U.S. Cl. CPC A61B 5/0809; A61B 5/7225; A61B 5/0044; A61B 5/0536

(57) ABSTRACT

An electrical impedance tomography (EIT) device (30) with an electrode array (33), with a measured value acquisition and feed unit (40), with a computing/control unit (70) and with a data input unit (50). The computing/control unit (70) coordinates the operation and the data acquisition of EIT data (3) and is configured to identify a heart region.

### CROSS REFERENCE TO RELATED APPLICATIONS

This application claims the benefit of priority under 35 U.S.C. § 119 of German Application 10 2018 008 545.8, filed Nov. 1, 2018, the entire contents of which are incorporated herein by reference.

### TECHNICAL FIELD

The present invention pertains to a device and to a process for electrical impedance tomography (EIT) with identification of a heart region.

### TECHNICAL BACKGROUND

Devices for electrical impedance tomography (EIT) are known from the state of the art. By means of an array of electrodes, these devices are configured and intended to generate an image, a plurality of images or a continuous image sequence by means of an image reconstruction algorithm from signals obtained by means of electrical impedance measurements and from data and data streams obtained from these.

These images or image sequences show differences in the conductivity of different body tissues, bones, skin, body fluids and organs, for example, of blood in the lungs and heart, as well as of breathing air in the lungs. As a result, it also becomes possible to visualize the skeletal structure surrounding the heart and the lungs (costal arches, sternum, spine) in a plane, the so-called transverse plane, in a horizontal tomogram, in addition to the heart and the lungs.

Thus, U.S. Pat. No. 6,236,886 describes an electrical impedance tomograph with an array of a plurality of electrodes, current feed to at least two electrodes and a process with an algorithm for image reconstruction for determining the distribution of conductivities of a body, such as bone, skin and blood vessels in a general configuration with components for signal acquisition (electrodes), signal processing (amplifiers, A/D converters), current feed (generator, voltage-current converter, current limiter) and control components (μC).

WO 2015/048917 A1 shows a system for electrical impedance tomography. The EIT system is suitable for detecting electrical properties of the lungs of a patient as impedances. Impedance values and impedance changes in the lungs are detected for this purpose by means of voltage or current feed between two or more electrodes and by means of a signal acquisition at an electrode array, usually in a continuous manner, and these values are subjected to further processing by means of data processing. The data processing comprises a reconstruction algorithm with a data processor in order to determine and reconstruct the electrical properties from the impedances. An anatomic model is selected from a plurality of anatomic models on the basis of biometric data of the patient during the reconstruction of the electrical properties from the acquired measured values, and the reconstruction of the EIT image data is adapted on the basis of the anatomic model or the biometric data.

It is explained in U.S. Pat. No. 5,807,251 that it is known in connection with the clinical application of EIT that a set of electrodes shall be provided, which are arranged in electrical contact with the skin at a defined distance from one another, for example, around the chest of a patient, and an electrical current or voltage input signal is applied alternatingly between different pairs or between all the possible pairs of electrodes, the electrodes being arranged adjacent to one another. While the input signal is applied to one of the pairs of electrodes arranged next to one another, the currents or voltages between each mutually adjacent pair of the rest of the electrodes are measured, and the measured data obtained are processed by means of an image reconstruction algorithm in order to obtain a visualization of the distribution of the specific electrical resistance over a cross section of the patient, around whom the electrode ring is arranged, and to display it on a display screen.

An impedance measurement is carried out on the chest by means of an electrode array around the chest of a patient with an EIT device, as it is known, for example, from U.S. Pat. No. 5,807,251, and an image of the lungs of the patient is generated from the impedances by means of a conversion to the geometry of the chest. With a total of, for example, 16 electrodes arranged around the chest of a patient, an EIT device can generate an image of the lungs with 32×32 pixels in one measuring run of current feeds at two respective electrodes each and by recording voltage measured values (EIT measured signals) at the other electrodes. A number of 208 impedance measured values are thus detected in the process at the electrodes in the case of the 16 electrodes. A set of 1024 pixels is then obtained with the EIT image reconstruction from these 208 impedance measured values.

The position in space and the extension in space of the heart in the thoracic cavity (chest) change in connection with breathing and ventilation because the position in space of the heart is influenced by the filling and emptying of the lungs with breathing/due to the removal of breathing gas. This happens, on the one hand, as an essentially cyclical vertical change in the position of the heart due to the contracting and relaxing movements of the diaphragm during the so-called abdominal breathing (abdominal type of breathing). However, there also is a change in the axial position of the heart due to dilation and narrowing of the area of the chest or thorax by means of the diaphragmatic muscles during the so-called diaphragmatic breathing (costal type of breathing). In addition, there are continuous changes in the circumference of the thorax in case of both costal breathing and abdominal breathing especially in the area of the costal arches due to filling and emptying of the lungs cyclically with breathing and/or ventilation. This results in the situation that due to breathing and/or ventilation and the type of breathing (abdominal breathing, costal breathing), the spatial and local composition of the tissue types located within a detection area of the electrode array is influenced in terms of both position (vertical, axial), extension (circumference of the thorax, circumference of the chest) and type (lungs, heart).

Depending on the positioning of the electrode array on the thoracic circumference, lung tissue as well as lung tissue and heart tissue are present in the area of the horizontal plane of the electrode plane, which is noticeable in the impedance values acquired by means of the electrical impedance tomography (EIT).

In case the electrode array is positioned on the thoracic circumference in the area of the fourth to sixth intercostal spaces, the acquired impedance values, which are representative of regions of the heart and lungs in the thoracic space, are present. Contrary to this, the acquired impedance values are representative of the regions of the heart and lungs in the thoracic space in another manner or to a lesser extent if the electrode array is positioned on the thoracic circumference in the area below the sixth to seventh intercostal spaces.

### SUMMARY

An object of the present invention is to provide an electrical impedance tomography device and a process for electrical impedance tomography for identifying a position in space of a heart region in relation to regions of the lungs in the thorax of a patient.

Another object of the present invention, which object is closely linked with the aforementioned object, is to propose a device and a process with which the heart region is taken into consideration during the analysis and visualization of electrical impedance tomography images of the thorax of a patient.

Another object of the present invention, which object is closely linked with the aforementioned object, is to propose a device and a process for identifying and providing a position of an electrode array, which is suitable for electrical impedance tomography and is arranged on the thorax of a patient.

Features and details that are described in connection with the process according to the present invention also apply, of course, in connection with and in respect to the device suitable for carrying out the process and vice versa, so that reference is and can always mutually be made to the individual aspects of the present invention concerning the disclosure.

Advantageous embodiments of the present invention appear from the subclaims and will be explained in more detail in the following description, partially in reference to the figures.

Furthermore, the process may also be provided as a computer program or a computer program product, so that the scope of protection of the present application also extends to the computer program product and to the computer program.

According to the present invention data (EIT data) obtained by means of an electrical impedance tomography device are processed in such a manner that an analysis in respect to a position of an electrode array on the thorax of a patient is made possible. The electrode array has a plurality of electrodes, which are arranged at spaced locations from one another around the circumference of the body in the area of the thorax of a living being. The electrode array is arranged horizontally on or around the thorax of a patient. At least two of the electrodes of the electrode array are configured for feeding an alternating current or an alternating voltage, and at least two of the other electrodes of the electrode array are configured for acquiring measured signals. The electrical impedance tomography (EIT) is able to differentiate between lung tissue and tissues of the heart and blood vessels in a spatially resolved manner from impedance differences between air/gas and blood.

A position in space of a heart region is determined in relation to regions of the lungs in the thorax of a patient. The position in space of the heart region is variable in time and space in the rhythm of breathing and/or ventilation. Depending on the current situation of the patient's spontaneous breathing (phases of spontaneous inhalation and phases of spontaneous exhalation) or of the mechanical ventilation with mechanical, purely mandatory ventilation modes (phases of mechanical mandatory inhalation and phases of mechanical mandatory exhalation) or with assisting ventilation modes in case of partial breathing activity of the patient (phase of spontaneous or patient-induced inhalation, phase of spontaneous or patient-induced exhalation), the heart becomes displaced due to the alternation of inhalation and exhalation. Moreover, the extension in space of the heart region due to systole (contraction) and diastole (relaxation) in the rhythm of the heartbeat (heart rate) is variable. Another effect on the image region of the heart, which is visible in the EIT, arises from the positioning of the patient (dorsal position, prone position, lateral position) as well as from changes in position, e.g., from the dorsal position to the lateral position and vice versa. In addition, the extent to which the heart region is visible in the EIT is affected by the height of the electrode array, which is placed on the chest and which is configured, for example, in the form of an electrode belt. The position in space of the heart region in the area of the thorax can be identified by checking by means of an analysis performed with a data processing whether and where areas with impedances and impedance time curves that are not typical for lung tissue but are typical of the type of tissues of the heart and blood vessels occur in the measured detection areas of the electrode array on the thorax next to areas with impedance values, impedance changes and/or impedance time curves that are typical of lung tissue. The measured detection area of the electrode array with the use of electrical impedance tomography (EIT) on the thorax is typically obtained as a horizontal plane at the level of the plurality of electrodes arranged around the chest of the patient, and the impedance values detected by means of the electrode array partially also include the properties of the tissues of regions located about 0.02 m to 0.1 m above as well as below and parallel to the electrode array around the chest of the patient. The electrode array makes possible a so-called transverse view to the thorax of the patient, i.e., a horizontal sectional view in the plane of the electrodes arranged on the thorax. This horizontal sectional view, which can be visualized by means of EIT, is a projection of the conductivity distributions in the entire region of the heart and lungs in the thorax, and the conductivity changes that are located at a greater distance from the EIT electrode plane are weighted in the projection with a lower weight with increasing distance from the EIT electrode plane than are the conductivity changes that are located in or close to the EIT electrode plane. In an expanded configuration of the electrode array, an electrode array with at least two electrodes, which are arranged at vertically spaced locations in horizontal planes, can be used, for example, instead of an electrode belt with which a plurality of electrodes can be applied or arranged in only one horizontal plane around the thorax of the patient. Such a configuration will be called “electrodes in two electrode planes” in a simplified manner in the further course of this application. For example, a three-dimensional EIT imaging (3D-EIT) can be made possible by means of such a plurality of electrodes arranged in at least two—or more than two—horizontal planes. Such an arrangement of electrodes in at least two electrode planes can be used to determine the position in space of the heart region in the area of the thorax. If the vertical distance between the two electrode planes is known, this distance information can also be included in the determination of the position in space of the heart region in the area of the thorax. Such an arrangement may be configured, for example, as a configuration of two separate electrode belts, as well as as a kind of piece of clothing being worn especially on the chest, quasi as an electrode vest with two integrated electrode belts or two rows of a plurality of electrodes each, which rows are arranged at horizontally spaced locations from one another. A known distance is obtained now between the two horizontal electrode planes especially in the case of the aforementioned special piece of clothing worn on the chest, so that this distance information can advantageously be included in both the determination of the position in space of the heart region in the area of the thorax and in the determination of the position of the electrode array arranged on the thorax. This distance information of the two electrode planes from one another is especially advantageous for determining the position especially for the determination of a horizontal position of these two electrode planes in relation to the position of the heart as well as in relation to the position of the lungs. It can happen in case of a double electrode belt, in which the two electrode planes are arranged at a defined vertical distance in relation to one another, in the case of a vertical axial rotation of the double electrode belt on the thorax that significant elements, for example, the outer contours of the lungs, or distinctive partial sections of the outer contours of the lungs, will be markedly shifted in relation to one another in the EIT image data of the two electrode planes. If the double electrode belt is arranged in too low a position vertically on the thorax/torso, it may happen that the position of the heart cannot be identified in the EIT in the EIT image data in one of the two electrode planes. This can be analyzed as a basis for an output signal, which will then indicate the incorrect vertical position of the double electrode belt on the thorax. The output signals can be used to provide indications and/or corresponding instructions for actions for the user. Including the known, defined distance of the two electrode planes, the indication may be expanded to indicate the distance by which the double electrode belt was arranged too low on the thorax/torso. The heartbeat cycles have a certain variability in the heartbeat/heart rate and are asynchronous with the breathing and are different from the respiration rate. There are a plurality of heartbeat cycles at the same time during one breath of a patient. Blood flows into the lungs and also out of the lungs with each heartbeat, which is visualized in different manners in different local regions and partial regions, the so-called ROI (Region of Interest) in the impedance values, impedance changes and impedance curves and can also be made visible in EIT visualizations and EIT images of the thorax of a patient in the time curve of breathing and/or heartbeat cycles. EIT measured signals or EIT raw data, which were acquired and obtained as EIT data by means of an electrical impedance tomography device (EIT device) and are provided by this device, can be used for the further data processing to distinguish different regions (lungs, heart) in the thorax of the patient. Furthermore, EIT image data, which were acquired and obtained as EIT data by means of an electrical impedance tomography device (EIT device) and are provided by this device, may be used for the further data processing.

The following signals or data, which can be detected with an EIT device by means of a group of electrodes or by means of an electrode belt, shall be defined as EIT measured signals or EIT raw data in the sense of the present invention. These include EIT measured signals and EIT data with different signal characteristics, such as electrical voltages or voltage measured signals, electrical currents or current measured signals, assigned to electrodes or to groups of electrodes or to positions of electrodes or of groups of electrodes on the electrode belt, as well as electrical resistance or impedance values derived from voltages and currents. EIT image data are defined in the sense of the present invention as data that were determined with a reconstruction algorithm from the EIT measured signals or EIT raw data and local impedances, impedance differences or impedance changes of regions of the lungs or of regions of the lungs and of the heart of a patient. The EIT data may be limited to a certain observation period or may have been derived as a subset of a data set of impedance values, which data set was acquired over a longer time period, or of values or data derived from impedance values. The observation period may arise here in connections with breathing and/or ventilation, for example, as time periods with continuous phases of inhalation and phases of exhalation or also as time periods with a plurality of phases of inhalation or of phases of exhalation.

The data processing of the EIT data is structured in the following manner and is carried out in the process according to the present invention for operating an electrical impedance tomography (EIT) device and in the electrical impedance tomography (EIT) device according to the present invention by means of a coordinated interaction of a data input unit, of a data output unit and of a computing and control unit in order to identify a current position in space of a heart region in relation to regions of the lungs in the thorax in an automated manner:

provision of a data set of EIT data,

determination of a first data set with data that indicate spatial and local distributions of impedance values and/or impendence changes of regions of the lungs in the thorax on the basis of the data set of EIT data,

determination and provision of a first output signal, which indicates a current position in space of regions of the lungs in the thorax on the basis of the data set of EIT data as well as on the basis of the first data set,

determination of a second data set with data, which indicates spatial and local distributions of impedance values and/or impedance changes of regions of the heart in the thorax on the basis of the data set of EIT data, and

determination and provision of a second output signal, which indicates a current position in space of a heart region in relation to regions of the lungs in the thorax on the basis of the data set of EIT data as well as on the basis of the second data set.

In a process according to the present invention for operating an electrical impedance tomography (EIT) device, a first data set of spatial and local distributions of impedance values and/or impedance changes of regions of the lungs in the thorax and a determination of a second data set of spatial and local distributions of impedance values and/or impedance changes of regions of the heart in the thorax are carried out after the provision of a data set of EIT data on the basis of the data set of EIT data. In the process according to the present invention for determining a position in space of a heart region in relation to regions of the lungs in the thorax, the previously described structure of the data processing is preferably implemented as a sequence of steps:

### Step 1:

Provision of a data set of EIT data,

### Step 2:

Determination of a first data set on the basis of the data set of EIT data. The first data set indicates spatial and local distributions of impedance values and/or impedance changes of regions of the lungs in the thorax,

determination and provision of a first output signal on the basis of the data set of EIT data as well as on the basis of the first data set. The first output signal indicates a current position in space of regions of the lungs in the thorax.

### Step 3:

Determination of a second data set with data on the basis of the data set of EIT data. The second data set indicates spatial and local distributions of impedance values and/or impedance changes of regions of the heart in the thorax, and

determination and provision of a second output signal on the basis of the data set of EIT data as well as on the basis of the second data set. The second output signal indicates a current position in space of a heart region in relation to regions of the lungs in the thorax.

The above-described structure of the data processing is implemented in the electrical impedance tomography (EIT) device according to the present invention by means of an interaction of a data input unit, of a data output unit and of a computing and control unit under the coordination of the computing and control unit. The data input unit, the data output unit and the computing and control unit are preferably arranged together with the electrode array, with other units, such as units for signal acquisition, signal amplification, signal filtering, units for voltage supply, units for data exchange (interface) and data management (network) as an EIT system with one another, but they may also be connected to one another and arranged as individual modules in a data network for interaction. The data input unit preferably has interface elements, for example, amplifiers, A/D converters, components for overvoltage protection (ESD protection), logic elements and other electronic components for the wired or wireless reception of data and signals, as well as adaptation elements, such as code or protocol conversion elements for adapting the signals and data for the further processing in the computing and control unit. The computing and control unit has elements for data processing, computing and sequential control, such as microcontrollers (μC), microprocessors (μP), signal processors (DSP), logic units (FPGA, PLD), memory components (ROM, RAM, SD-RAM) and combination variants thereof, for example, in the form of an “embedded system,” which are configured together with one another, are adapted to one another and are configured by programming to execute the process for operating an electrical impedance tomography (EIT) device. The data output unit is configured to generate and provide the output signal. The output signal is preferably configured as a video signal (e.g., Video Out, Component Video, S-Video, HDMI, VGA, DVI, RGB) to make possible a graphic, numeric or pictorial visualization on a display unit connected to the output unit in a wireless manner (WLAN, Bluetooth, WiFi) or in a wired manner (LAN, Ethernet) or on the output unit itself.

All the advantages that can be achieved with the process described can be achieved in the same manner or in a similar manner with the described device for carrying out the process and vice versa.

For the determination of a position in space of a heart region in relation to regions of the lungs in the thorax, the device according to the present invention for determining a position in space of a heart region in relation to regions of the lungs in the thorax has a data input unit, a computing and control unit and a data output unit, wherein the device

is configured by means of the data input unit to receive data and to provide a data set of EIT data,

is configured by means of the computing and control unit to process the data set of EIT data to determine a first data set with data that indicate spatial and local distributions of impedance values and/or impedance changes of regions of the lungs in the thorax and to process the first data set and the data set of EIT data to determine a first output signal, which indicates a current position in space of regions of the lungs in the thorax,

is configured by means of the computing and control unit to process the data set of EIT data to determine a second data set with data that indicate spatial and local distributions of impedance values and/or impedance changes of regions of the heart in the thorax to process the second data set and the data set of EIT data to determine a second output signal, which indicates a current position in space of a heart region in relation to regions of the lungs in the thorax, and

is configured by means of the data output unit to provide the first output signal and the second output signal.

Signal values that indicate impedance values and/or impedance changes of regions of the lungs in the thorax are often also called ventilation-induced signals or ventilation-related impedance changes (VRIC). Signal values that indicate impedance values and impedance changes of regions of the heart in the thorax are often also called heart-specific (cardiac-related impedance changes=CRIC) signals.

The determination of the first data set, which indicates spatial and local distributions of the impedance values and/or impedance changes of regions of the lungs in the thorax, based on the data set of EIT data, can be carried out in the following manner such that signals or signal components that can be assigned to a range of typical respiration rates based on the frequency spectrum are extracted from the data set of EIT data. One possibility of the extraction is made possible by the fact that the signal values in the EIT data, which indicate impedance values and/or impedance changes of regions of the lungs in the thorax (VRIC), have a signal amplitude that is greater by one order of magnitude than the cardiac-related signals (CRIC) and, for example, an extraction of the ventilation-related signals (VRIC) can thus be carried out by means of an application of threshold values. For example, a value of 50% of the arithmetic mean of all signal values of the EIT data over a defined time course or a value of 50% of a global impedance curve may be used as a threshold value that is suitable for this. A possibility for obtaining the global impedance curve from the EIT data is described, for example, in US 2016 354 007 A1 (US 2016 354 007 A1 is incorporated herein by reference). As an alternative to such an extraction, it is also possible to use a signal filtering. For example, a band-pass filtering with a transmission band of 0.1 Hz to 0.7 Hz may be used for this purpose, and low-pass filtering with a limit frequency of about 0.8 Hz may be used as an alternative or in addition to blank out signal components markedly above the typical frequency spectrum of the breathing activity of the patient, i.e., for example, frequency components in the range of the heartbeat in the range above approx. 1 Hz.

The determination of the second data set on the basis of the data set of EIT data can be carried out in the following manner such that signals or signal components that can be assigned concerning the frequency spectrum of spectral signal ranges above typical respiration rates are filtered out of the data set of EIT data by means of a high-pass filtering. The limit frequency of the high-pass filtering is selected here to be such that the second data set has essentially only signals with signal components in the frequency spectrum of the cardiac activity. An adapted high-pass filtering with a limit frequency in the range of 0.8 Hz to 2 Hz can make this possible. For example, a frequency range above a characteristic frequency of 0.67 Hz can be selected for the limit frequency in a physiologically meaningful range for an adult, which corresponds to a heartbeat rate of 40 beats per minute. For example, a frequency range above a characteristic frequency of 2 Hz can be selected for the limit frequency in a physiologically meaningful range for an approximately 2-year-old child, which corresponds to a heartbeat rate of 120 beats per minute. An application with high-pass/band-pass filtering is described in the scientific publication of Frerichs I, Pulletz S, Elke G, Reifferscheid F, Schadler D, Scholz J, Weiler N: “Assessment of changes in distribution of lung perfusion by electrical impedance tomography,” Respiration, 2009: pp. 3-4, as well as in the publication of Vonk Noordegraaf A, Kunst P W, Janse A, Marcus J T, Postmus P E, Faes T J, de Vries P M: “Pulmonary perfusion measured by means of electrical impedance tomography,” Physiology Measurements, 1998: pp. 265-267. The splitting of the data set of EIT data into the first and second data sets may also be carried out by averaging over time over a greater number of cardiac cycles, in addition to by the above-described low-pass, high-pass or band-pass filtering in the frequency range. As an alternative, the splitting of the data set of EIT data into the first data set and the second data set may also be carried out by means of methods that are based on the use of a principal component analysis, PCA. An application of the principal component analysis in connection with EIT data is described in the scientific publication of Deibele J M, Luepschen H., Leonhardt S: “Dynamic separation of pulmonary and cardiac changes in electrical impedance tomography.” Physiology Measurement, 2008, pp. 2 to 6.

The data set of EIT data and the first data set and the second data set are preferably addressed in the form of an index, and the data or impedance values detected on the EIT measuring channels, which indicate regions of the lungs and regions of the heart, are preferably addressed in the form of indicated vectors, indicated data fields or indicated matrices, stored, and kept available for the further processing (vector operations, matrix operations). This indication makes possible a spatially resolved assignment and addressing of individual data elements (pixels) or regions of a plurality of data points (ROI) of the data of the first and second data sets.

The determination of the first output signal is carried out by the first data set being selected as a subset of the data set of EIT data. The provision of the first output signal makes possible a representation or visualization of regions of the lungs, preferably in a transverse view, which illustrates the position, extension of pulmonary tissue in the thorax of the patient, as well as changes in the position and extension, as well as the quantity and quality of the ventilation of regions of the lungs with breathing gas in the course of the ventilation during the alternation of inhalation and exhalation.

The determination of the second output signal is carried out by selecting the second data set as a subset of the data set of EIT data. This selection with determination of the second data set and automated identification of the heart region with the determination of the second output signal takes place after the signal filtering such that the determination of the second data set is continued by calculating a power density spectrum for the mean signal of all impedance signals of all EIT image elements (pixels) in the data set of EIT data or in a subset of EIT image elements (pixels) in the data set of EIT data. The heart rate is determined in a characteristic frequency range by means of a robust method from this power spectrum or the power distribution or amplitude distribution derived herefrom. A range above a characteristic frequency of 0.67 Hz is obtained as a characteristic frequency range in a physiologically meaningful range for an adult, which corresponds to a heartbeat rate of 40 beats per minute. A characteristic frequency range is obtained in a physiologically meaningful range above a characteristic frequency of 2 Hz, for example, for an approximately 2-year-old child, which corresponds to a heartbeat rate of 120 beats per minute. A robust method is, for example, a parametric approach of an estimation by means of an autoregressive model, as it is described, for example, in a scientific paper by Takalo R.; Hytti H.; Ihalainen H.: “Tutorial on Univariate Autoregressive Spectral Analysis,” Journal of Clinical Monitoring and Computing, 2005, 19: pp. 402-404. The manner of the signal processing, especially the selection of the spectral analysis or transmission/blocking ranges of filters from the data set with information concerning the at least one cardiac function, can be derived especially on the basis of the heartbeat rate or the pulse of the heart, because typical heart rates differ from typical respiration rates by a factor of about 4 to 5. The determination of the heart rate from the data set of EIT data to determine the heart region can be carried out in an especially advantageous manner by means of a so-called Kalman filter. The mode of operation of a Kalman filter and the effect and advantages thereof in the signal processing are described in the scientific paper by Kalman R E: “A New Approach to Linear Filtering and Prediction Problems,” Transaction of the ASME, Journal of Basic Engineering, 1960, 82: pp. 35-45. Signal disturbances, caused, for example, by movement of the body, slight spontaneous breathing, simultaneous use of computed tomography, which occur uncorrelated to the measured signals, frequently occur during electrical impedance tomography. False-positive detections of blood volume pulses would be able to occur without the use of a suitable filtering. The Kalman filter is well suited for removing interference signals of this type and for providing a stable heart rate signal. The Kalman filter provides an output signal, which converges towards the interference-free value with an increase in the number of measured values, whose expected value corresponds to that of the interference-free signal, whose variance is minimized. The heart region is determined on the basis of the determined power distribution in the characteristic frequency range. The determination is carried out by selecting a region around the range of the maximum of the power or amplitude distribution, since the heart region is located in this region around the range of the maximum of this distribution. In addition to the power or amplitude distribution, an additional criterion may optionally and advantageously be applied when determining the second data set. This additional criterion requires that only signals of the same phase position be used in the second data set to determine the heart region. This leads to the advantage of an improved robustness of the data processing when identifying the heart region. The current position in space of the heart region is thus identified in relation to regions of the lungs in the thorax, and it can be used as the basis for the second output signal, which indicates the current position in space of the heart region in relation to regions of the lungs in the thorax. The provision of the second output signal makes it possible, for example, to represent or visualize the heart region, which illustrates the position and the extension of the heart in the thorax of the patient.

The use of the subset selected as the first data set from the EIT data with inclusion of the actual current heart region by means of the second output signal for the visualization as an EIT image of the thorax brings with it, unlike the use of the entire data set of EIT data, the advantage that the interpretability of the EIT image is not made difficult here by displacements of the position in space of the heart. Such displacements are induced by the respiratory movements.

The embodiments described below represent variations, variants of the data processing, which can complement or expand the sequence of steps of the process according to the present invention for operating an electrical impedance tomography (EIT) device, as well as the tasks of the computing and control unit in the electrical impedance tomography (EIT) device according to the present invention. These embodiments, hereinafter described, shall therefore also be defined concerning the disclosures as expansions in the functional scope, especially of the computing and control unit of the electrical impedance tomography (EIT) device according to the present invention. The advantages described for the process according to the present invention can be achieved in the same manner or in a similar manner with the device for carrying out the process according to the present invention, as well as with the described embodiments of the device. Furthermore, the embodiments described and their features and the advantages of the process can be extrapolated to the device, just as the described embodiments of the device can be extrapolated to the process. The data set of EIT data has signals or data belonging to at least one plurality of electrodes, which plurality of electrodes is arranged in a horizontal plane around the thorax.

In a special embodiment, the data set of EIT data may also have signals or data of at least two pluralities of electrodes, which pluralities are arranged parallel to one another and spaced apart at a defined distance.

Provisions are made in a preferred embodiment for the determination of a position of an electrode array on the thorax of a patient. In particular, provisions are made for determining a vertical position of the electrode array on the thorax, such as under the vertical. The electrode array may be configured, for example, as an electrode belt, which, adapted in size and length to the individual thoracic circumference of the particular patient, can be arranged optimally at the level of the fourth to sixth costal arch (ICS 5), in the area of the fourth to sixth intercostal space (ICS) (ICS 4 to ICS 6) around the chest of the patient. The position of the electrode array on the thorax of the patient is determined on the basis of the third data set. In this preferred embodiment, the computing and control unit is configured to determine and provide a control signal, which indicates the position of the electrode array on the thorax of the patient. The control signal is determined on the basis of the determined position of the heart. The control signal can be used to provide a user with a visual, acoustic or optical indication on whether or not the electrode array is positioned properly on the thorax of the patient. In case of proper positioning on the thoracic circumference as part of the data set of EIT data, the second data set, which indicates spatial and local distributions of the impedance values and/or impedance changes of regions of the heart in the thorax, is present in a defined order of magnitude. In case of a improper positioning, for example, closer to the abdominal circumference, the second data set, which indicates spatial and local distributions of the impedance values and/or impedance changes of regions of the heart in the thorax, is not present in a defined order of magnitude. For example, the position of the electrode array on the thorax of the patient can be determined such that quantity ratios in the data sets or region ratios in the EIT image between the first data set and the second data set are analyzed on the basis of a comparison variable relative to an EIT image, which images the current state of regions of the lungs and heart in the thoracic space both on the basis of data of the first data set and of data of the second data set. For example, an area equivalent of the second data set area of the heart, indicating the first data set area of the lungs, equaling less than 10%, could be considered to mean that the electrode array is not positioned correctly, i.e., for example, not on the thoracic circumference, but on the abdominal circumference. The control signal may also be used for an output to a display unit connected directly or indirectly to the EIT device, and for transmission into a data network (LAN, WLAN, PAN, Cloud).

In another preferred embodiment, the calculating and control unit is configured to carry out a continuous determination of the second data set and to take into account the second data set with data that indicates the spatial and local distributions of the impedance values and/or impedance changes of regions of the heart in the thorax during a data processing of the EIT data, which follow in time and are provided continuously, by the computing and control unit. The computing and control unit is configured here to take into account the previously determined second data set with data, which indicate the spatial and local distributions of the impedance values and/or impedance changes of regions of the heart in the thorax, or the current position in space of the heart region in relation to the regions of the lungs in the thorax during the determination of the first data set with data that indicate spatial and local distributions of impedance values and/or impedance changes of regions of the lungs in the thorax. Possible types of embodiment of such considerations are, for example, the blanking out of data or also markings, carried out, for example, as masking of data. The data that belong to the second data set are now marked, masked or blanked out by the computing and control unit in the data set of the EIT data in order to take them into account during the image reconstruction, during calibrations at start-up or during recalibrations during the operation, which may be necessary, for example, in case of repositioning of the patient or repositioning of the belt. Masking within the EIT data or blanking out of subsets of EIT data may be carried out both in the form of not taking the EIT data in question into account, and, as an alternative, the masking or blanking out of the corresponding EIT data may be carried out by equivalent data, for example, data of adjacent regions. The masked subsets may advantageously be copied into another data set or the remaining data, which are not blanked out, may be copied into another data set. Since impedance changes in the heart region, induced by the displacement of the heart in the breathing or ventilation cycle, lose some influence on this reference variable due to masking, the masking may be advantageous for the determination of reference variables, for example, for the global impedance curve calculated from the EIT data, i.e., the sum of the relative impedance changes over both regions (left lobe, right lobe) of the lungs or also for regional impedance curves, i.e., the sums of impedance changes within selected regions (ROI, Regions of Interest) of individual regions of the lungs within the thorax, if additional determined parameters can be determined with improved accuracy on the basis of these reference variables during the operation of the electrical impedance tomography (EIT) device. The functional EIT visualizations for ventilation, but also parameters derived therefrom, for example, the intratidal redistribution (ITV), the regional ventilation delays (RVD), in which the global impedance curve and/or the regional impedance curves are included as reference variables or mean values, undergo improvements in meaningfulness and accuracy, because subsets with data that belong to the heart region are not included as impedance changes synchronous with ventilation in regions of the heart region in the global impedance curve or regional impedance curves of certain regions (ROI), nor in other derived parameters (e.g., RVD, ITV). In addition, visualizations concerning the perfusion of the lungs and the pulse of the lungs may thus also undergo improvements in meaningfulness and accuracy. In principle, a large number of the functional EIT images with visualizations of ventilation, pulsatility and perfusion benefit from possibilities of marking, masking or blanking out of the EIT data, which are given with the present invention.

In another preferred embodiment, an adaptation of data processing and/or signal filtering can be performed for the EIT data provided chronologically later on the basis of the second data set. Adaptations of the limit frequency of the high-pass filtering can be derived from the frequency ranges of the cardiac activity, which frequency ranges can be determined from the second data set. For example, such determination of frequency ranges may take place at the beginning or after a high-pass prefiltering, for example, in a frequency range of about 0.5 Hz to 1 Hz, and a finer filtering, adapted to the range of the respective current heart rate of the particular patient, can be made possible in the further time course of the data processing.

In another preferred embodiment, the determined position of the heart region can be taken into account in a visualization of the EIT data. It is thus possible, preferably in a transverse view of the lungs, to visualize the heart in a prominent manner as a region. This is possible, for example, by visualization with different shades of gray, colors or patterns of regions of the heart and of regions of the lungs.

In another preferred embodiment, it is possible jointly to use information concerning the heart rate from external data sources, such as a physiological patient monitor, a blood pressure-measuring device, a measuring device for measuring the oxygen saturation (SPO₂), an EKG-measuring device or a diagnostic device, cardiography device or plethysmography device, which provides in any way a signal or data that indicates or also comprises a heart rate to adapt the limit frequency of the high-pass filtering.

In themselves as well as in combination with one another, the embodiments described represent special embodiments of the electrical impedance tomography device according to the present invention and of the electrical impedance tomography process according to the present invention for determining a position in space of a heart region in the area of the thorax in relation to regions of the lungs of a patient. Advantages arising from the combination or combinations of a plurality of embodiments and further embodiments are likewise covered by the inventive idea, even if all the possible combinations of embodiments herefor are not particularly described in detail. The above-described embodiments according to the present invention of the process may also be configured in the form of a computer-implemented process as a computer program product with a computer, wherein the computer is prompted to execute the above-described process according to the present invention when the computer program is executed on the computer or on a processor of the computer or on a so-called “embedded system” as part of a medical device, especially of the EIT device. The computer program may also be stored on a machine-readable storage medium. In an alternative embodiment, a storage medium may be provided, which is intended for storing the above-described, computer-implemented process and can be read by a computer. It is within the scope of the present invention that it is not absolutely necessary to execute all steps of the process on one and the same computer, but they may also be executed on different computers, for example, in a form of the cloud computer described in detail before. The sequence of the process steps may possibly be varied as well. Furthermore, it is possible that individual sections of the above-described process can be carried out in a separate unit, which is, for example, commercially available in itself, e.g., on a data analysis system arranged in the vicinity of the patient, and other parts can be carried out on a display and visualization unit, which is arranged, for example, as a part of a hospital information system preferably in a room set up for monitoring a plurality of hospital rooms, quasi as a distributed system.

The present invention will be explained now in more detail by means of the following figures and the corresponding descriptions of the figures without limitation of the general inventive idea. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of an arrangement of an EIT device with an electrode array;

FIG. 2a is a schematic view of arrays of electrodes according to FIG. 1;

FIG. 2b is a schematic view of arrays of electrodes according to FIG. 1;

FIG. 3a is a view of visualizations according to FIG. 2a;

FIG. 3b is a view of visualizations according to FIG. 2b;

FIG. 4 is a view of another visualization;

FIG. 5 is a schematic view of a flow chart for determining a heart region with determination of an electrode position; and

FIG. 6 is a schematic view of a flow chart for determining a heart region with determination of an electrode position.

### DESCRIPTION OF PREFERRED EMBODIMENTS

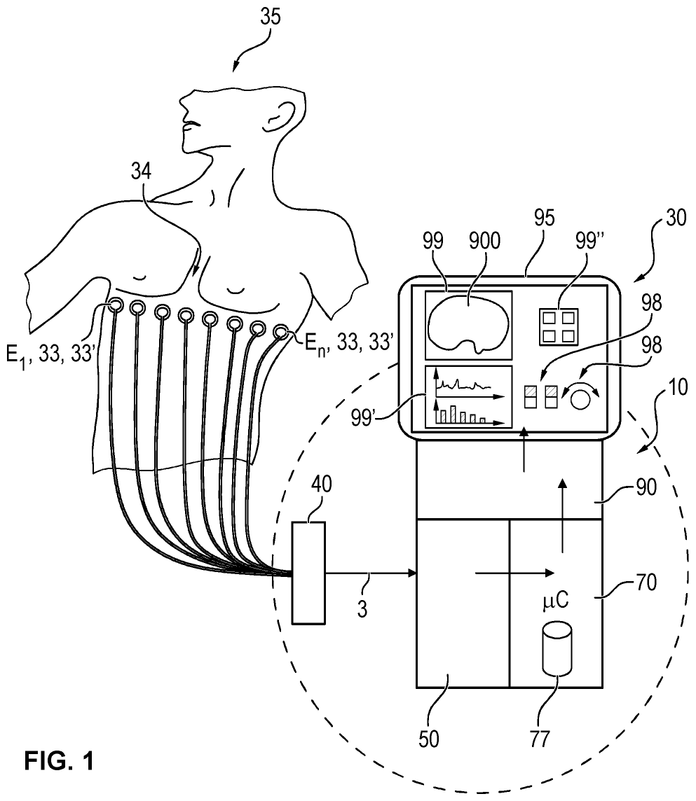

Referring to the drawings, FIG. 1 shows a schematic view of a device 10 for processing EIT data 3 from an EIT device 30 and of an electrode array 33 with a plurality of electrodes E₁, . . . Eₙ 33′. The electrode array 33 with the electrodes E₁, . . . Eₙ 33′ is arranged on the upper body (thorax) 34 of a patient 35. A measured value acquisition and feed unit 40 is configured to feed during a measuring cycle a signal, preferably an alternating current (current feed) or also an alternating voltage (voltage feed) to a respective pair of electrodes 33′. The voltage signals resulting from the alternating current feed are detected as signals at the other electrodes 33′ by the measured value acquisition and feed unit 40 and are made available as EIT data 3 for the data input unit 50. The EIT data 3 provided are fed in the EIT device 30 to a control unit 70 via a data input unit 50. A memory 77, which is configured to store a program code, is provided in the control unit 70. The running of the program code is coordinated by a microcontroller arranged in the control unit as an essential element or by another configuration of computing elements (FPGA, ASIC, μP, μC, GAL). The computing and control unit 70 is thus prepared and intended for coordinating the operation of the EIT device 30 and to execute the described steps with comparison operations, computation operations, storage and data organization of the data sets. The values determined by the control unit 70 are visualized on a display device 95 by means of a data output unit 90. Additional elements 99′, for example, operational controls 98, elements 99″ for visualizing numerical values or elements 99′ for visualizing time curves or curves, are also present on the display device 95 in addition to the visualization 900.

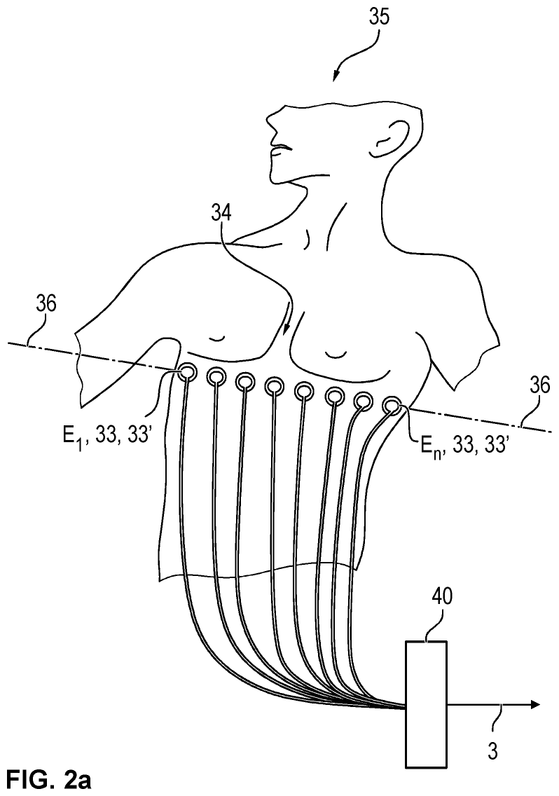

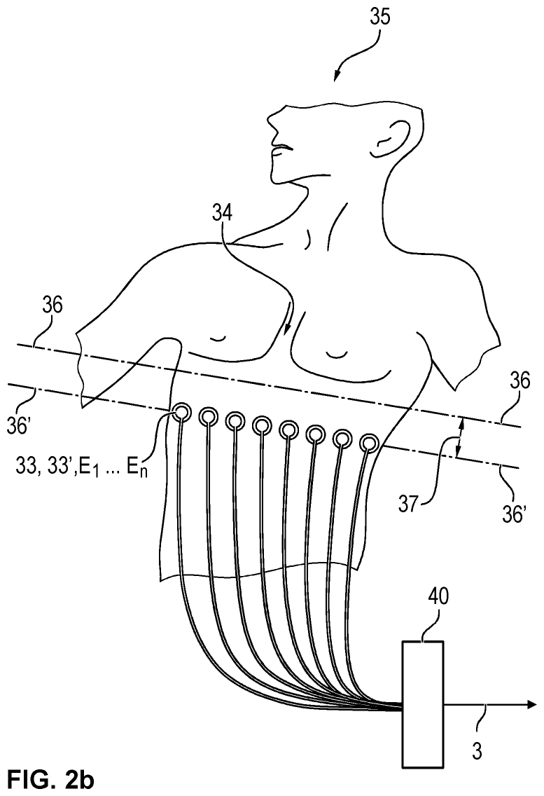

FIGS. 2a and 2b show views of different arrangements of electrode arrays 33 on the thorax 34 according to FIG. 1. Identical elements in FIGS. 1, 2a, 2b are designated by the same reference numbers in FIGS. 1, 2a and 2b. FIG. 2a shows a first arrangement of the electrode array 33 and electrodes 33′ on the thorax 34 according to the schematic view shown in FIG. 1 in a horizontal normal position 36. FIG. 2b shows a second arrangement of the electrode array 33 and electrodes 33′ on the thorax 34 according to the schematic view shown in FIG. 1 in a horizontal position 36′. A horizontal deviation 37 between the normal position 36 and the deviating position 36′ is shown.

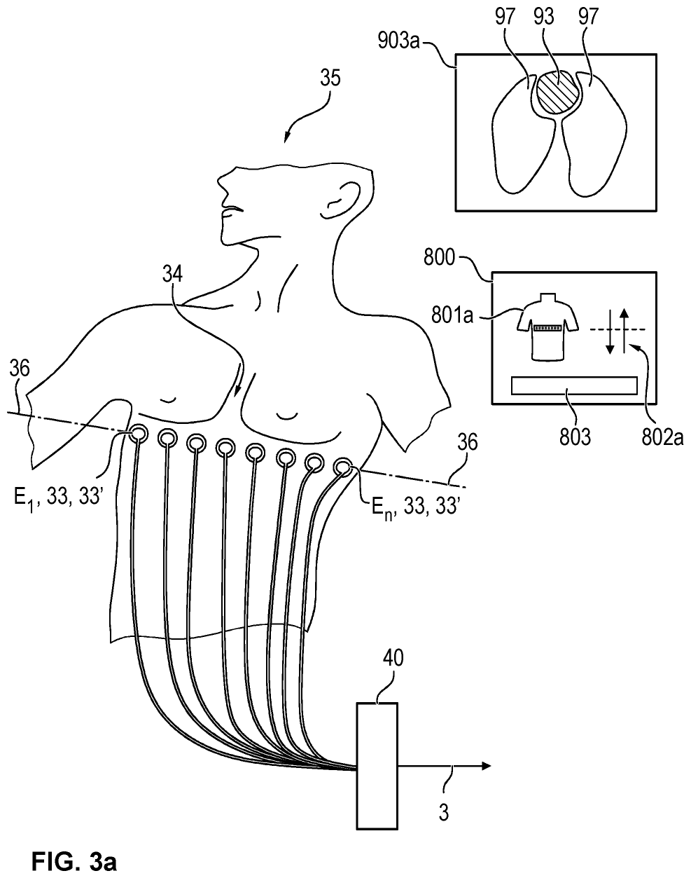

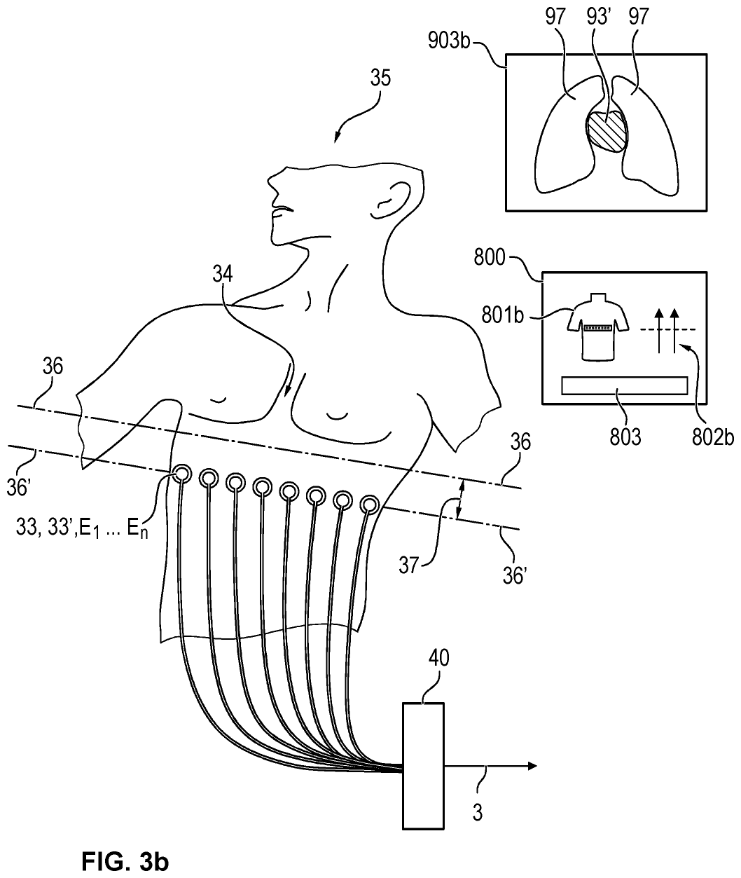

FIGS. 3a and 3b show views of visualizations according to the arrangements shown in FIGS. 2a and 2b. Identical elements in FIGS. 1, 2a, 2b, 3a, 3b are designated by the same reference numbers in FIGS. 1, 2a, 2b, 3a and 3b. FIGS. 3a and 3b show corresponding visual representations 903a, 903b of the visualization 900 (FIG. 1) on the display device 95 (FIG. 1) for the positions 36, 36′ of the electrodes 33, 33′ on the thorax 34 according to FIGS. 2a and 2b. The effects of different vertical positions 36, 36′ of the electrodes 33, 33′ on the thorax 34 on the visualization 900 (FIG. 1) in the visual representations 903a, 903b are shown here. These FIGS. 3a and 3b show in the visual representations 903a, 903b the heart region 93, 93′ and the lung regions 97, 97′ in transverse views and in a schematic manner. In addition to the visualization 900 (FIG. 1), graphic representation elements 801a, 801b, for example, in the form of arrow representations 802a, 802b, which shall symbolize the current positions 36, 36′ of the electrode array 33 on the thorax 34 and the necessary corrections of the electrode array 33 on the thorax 34, are arranged in a separate symbolic representation 800 as an optional embodiment of the elements 99, 99′, 99″ (FIG. 1) of the display device 95 (FIG. 1). In addition, an output field 803 is provided, which is intended to provide the user with a text message, in addition to the arrow representations 802a, 802b, concerning a correct arrangement—according to FIG. 2a and FIG. 2b—of the electrode array 33, 33′ on the thorax or concerning an incorrect arrangement, i.e., arrangement in an excessively low position—according to FIG. 2b and FIG. 3b—of the electrode array 33, 33′ on the thorax 34. For example, the horizontal deviation 37 can be outputted in this output field 803 for the user for orientation, and additional indications or suggestions for actions to be taken may be outputted therein as well.

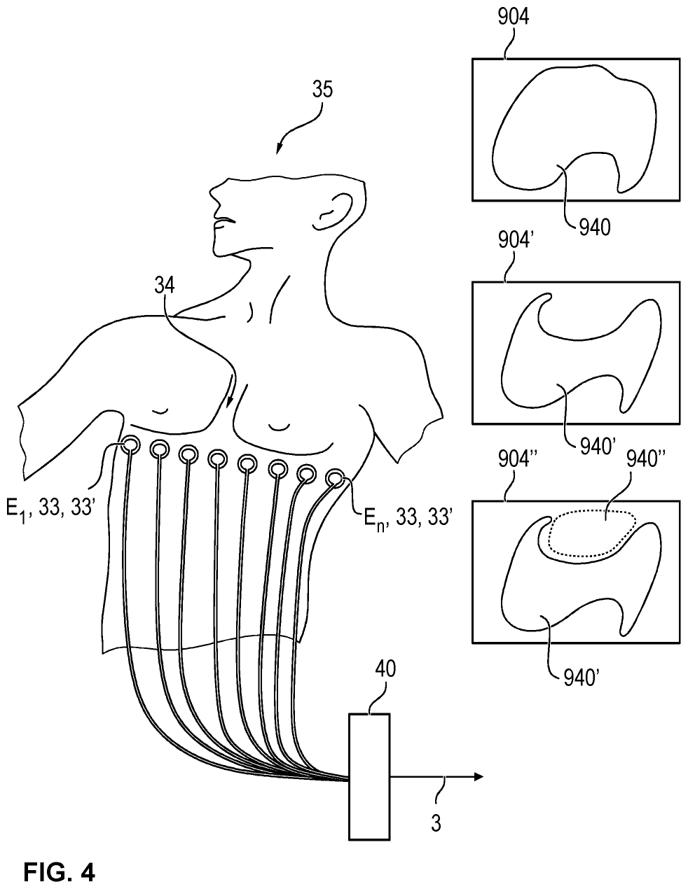

FIG. 4 shows two different variations 904, 904′, 904″ of representations of visualizations 900 (FIG. 1) of EIT images without a position of the heart region in relation to regions of the lungs being taken into account and with a position of the heart region in relation to regions of the lungs being into account. Identical elements in FIGS. 1, 2a, 2b, 3a, 3b, 4 are designated by the same reference numbers in FIGS. 1, 2a, 2b, 3a, 3b and 4. The representation 904 shows an EIT image 940 of regions of the lungs, in which the heart region was not included in the formation of the representation. The representation 904′ shows an EIT image 940′, in which the heart region was also included in the formation of the representation, by image regions (pixels) belonging to the heart region being represented in this EIT image 940′ next to the regions of the lungs as regions without any information, i.e., the corresponding regions are “blanked out” in the EIT image 940′. The image regions (pixels) that belong to the heart region are shown in the representation 904″ as an independent image region 940″ separated from regions of the lungs.

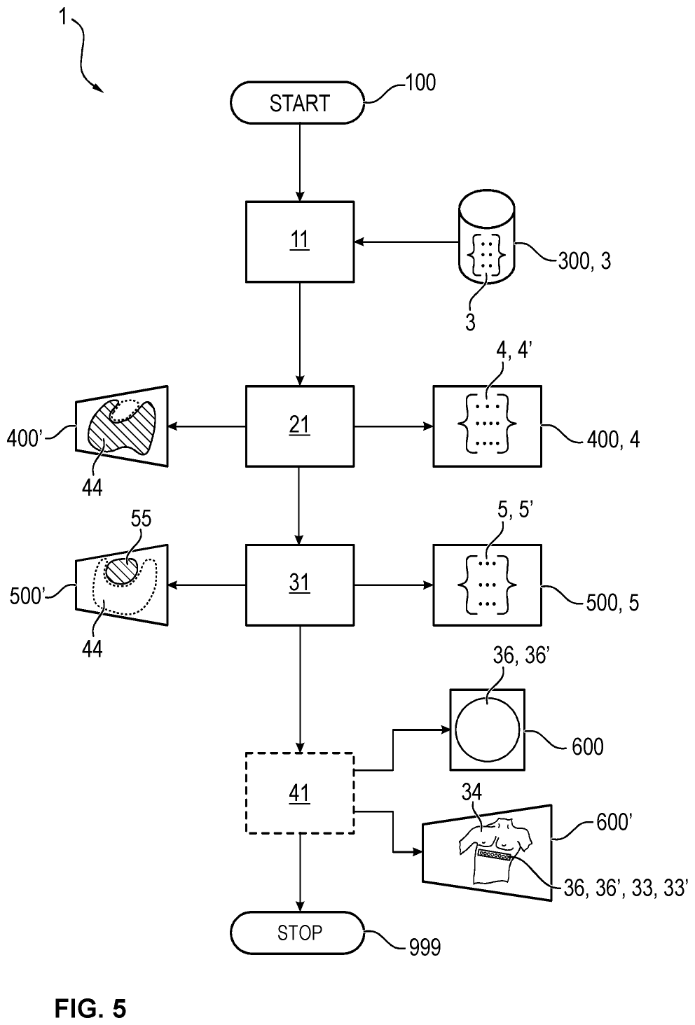

FIG. 5 shows a flow chart, which shows a sequence 1 for processing data 3 obtained by means of an electrical impedance tomography (EIT) device 30 (FIG. 1) to determine a position in space of a heart region in relation to regions of the lungs in the thorax of a patient. Identical elements in FIGS. 1, 2a, 2b, 3a, 3b, 4 and 5 are designated by the same reference numbers in FIGS. 1, 2a, 2b, 3a, 3b, 4 and 5.

The processing is shown on the basis of a sequence 1 of steps, which begins with a start 100 and ends with a stop 999.

A data set 300 of EIT data 3 is provided in a first step 11.

A first data set 400 with data 4, which indicates spatial and local distributions of the impedance values and/or impedance changes of regions of the lungs in the thorax 34 (FIG. 1), is determined on the basis of the data set 300 of EIT data 3 in a second step 21. In addition, a first output signal 400′, which indicates a position 400 in space of regions of the lungs in the thorax 34 (FIG. 1), is provided in the second step 21 on the basis of the data set 300 of EIT data 3 as well as on the basis of the first data set 400. The determination of the first data set 400 is carried out on the basis of the signal values that indicate impedance values and/or impedance changes of regions of the lungs in the thorax 34 (FIG. 1) on the basis of a data extraction or data filtering from the data set 300 of EIT data 3. The data extraction may be carried out, for example, on the basis of an amplitude analysis or by means of a threshold value comparison of the signal amplitudes of the EIT data 3, which is made possible by the signal values in the EIT data 3, which indicate impedance values and/or impedance changes of regions of the lungs 97 (FIG. 4), having a signal amplitude that is greater by an order of magnitude than the cardiac-related signals. An alternative possibility arises from a use of frequency-specific signal filtering, for example, with a low-pass filtering with a limit frequency above 0.8 Hz (adults) or above 2 Hz (infants). It should be noted in this connection that due to the rhythmic filling and emptying of the lungs with breathing gases and to the associated movement and displacement of the heart relative to the lungs and within the thorax 34 (FIG. 1), regions in the thorax (FIG. 1), in which impedance changes caused actually directly by the rhythmic alternation of inhalation and exhalation due to ventilation-induced changes of state are present, are also represented in the first data set 400, but regions in which impedance changes taking place synchronously with ventilation are caused by displacements of the lungs and heart in space cannot be distinguished from these regions. When using this first output signal 400′ for a visual output of an EIT image with representation of the position 44 in space of the lungs in the thorax 34 (FIG. 1), the regions of the heart in the thorax 34 (FIG. 1) cannot yet be visualized in a differentiated manner. A further analysis, as it will be continued in the further, third step 31, is required for this.

A second data set 500, which indicates the spatial and local distributions of impedance values 5 and/or impedance changes 5′ of regions of the heart in the thorax 34 (FIG. 1), is determined in a third step 31 on the basis of the data set of EIT data. A second output signal 500′, which indicates a position 55 in space of the heart in relation to the regions 44 of the lungs in the thorax 34 (FIG. 1), is provided in the third step 31 on the basis of the data set 300 of EIT data 3 as well as on the basis of the second data set 500. The determination of the second data set 500, which indicates spatial and local distributions of the impedance values 5 and/or impedance changes 5′ of regions of the heart in the thorax 34 (FIG. 1), may be carried out, for example, by means of an adapted high-pass filtering of the data set 300 of EIT data 3 with a limit frequency in the range of 0.8 Hz to 2 Hz.

An additional data set 600, which indicates a position 36, 36′ of the electrode array 33 on the thorax 34 (FIG. 1) of the patient 35 (FIG. 1), is determined in an optional, fourth step 41 on the basis of the data set 300 of EIT data 3 as well as on the basis of the second data set 500. A control signal 600′, which indicates the position 36, 36′ of the electrode array 33 on the thorax 34 (FIG. 1), is provided in the optional, fourth step 41 on the basis of the additional data set 600.

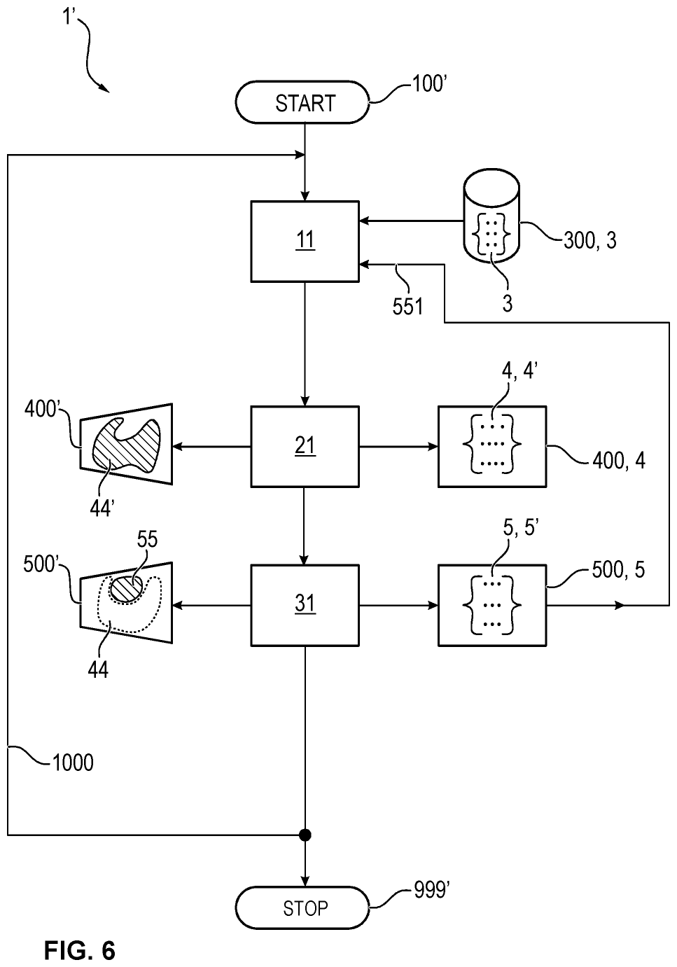

FIG. 6 shows a flow chart, which shows a sequence 1′ for a processing of data 3 obtained by means of an electrical impedance tomography (EIT) device 30 (FIG. 1) to determine a position in space of a heart region in relation to regions of the lungs in the thorax 34

(FIG. 1) of a patient. Identical elements in FIGS. 1, 2a, 2b, 3a, 3b, 4, 5, 6 are designated by the same reference numbers in FIGS. 1, 2a, 2b, 3a, 3b, 4, 5, 6. The processing is shown on the basis of a sequence of steps 1′, which begins with a start 100′ and ends with a stop 999′ and is largely identical to the sequence 1 described in connection with FIG. 5. This sequence 1′ according to this FIG. 6 is expanded compared to the sequence 1 according to FIG. 1 to the extent that, on the one hand, the data provision of the EIT data 3 as well as the data processing (sequence of steps 11, 21, 31) with the determination of the first data set (400) and of the second data set (500) and of the output signals (400′, 500′) belonging to these data sets and of the identified regions of the lungs 44 and of the determined position in space of the heart 55 take place continuously over time. This is illustrated by the return branch 1000 of stop 900′ to the start 100′ in FIG. 6.

A further expansion of the sequence 1′ compared to the sequence 1 (FIG. 5) arises from the fact that the second data set 500 of the provided data set 300 of EIT data 3 is provided during the continuous data provision and data processing. This is illustrated by the signal path 551 in FIG. 6. The second data set 500 can thus be used to mark, mask or blank out subsets in the data set of EIT data 3 in order to derive, on the one hand, continuously improved visualizations of regions of the lungs 44′ from the EIT data 3 in the further time course of the EIT application by blanking out the heart region 55 and to display them on a display device (FIG. 1) and, on the other hand, to determine some parameters, for example, the global impedance curve, which is commonly used in EIT, with improved accuracy. The improved accuracy of the global impedance curve arises from the circumstance that impedance changes of regions of the heart region 55, which changes are synchronous with the ventilation, cannot be included by the computing and control unit 70 (FIG. 1) in the calculation of the global impedance curve. What was stated concerning the global impedance curve also applies in a comparable manner to additional parameters, such as RVD, ITV and visualizations 900 (FIG. 1) of ventilation, pulsatility and perfusion. The optional, fourth step 41 shown in FIG. 5 and data sets 600 and the control signals 600′ obtained in this connection are not shown in FIG. 6 for the sake of clarity.

While specific embodiments of the invention have been shown and described in detail to illustrate the application of the principles of the invention, it will be understood that the invention may be embodied otherwise without departing from such principles.

### LIST OF REFERENCE NUMBERS

1 Sequence

3 EIT data

4 Impedance values of regions of the lungs

4′ Impedance changes of regions of the lungs

5 Impedance values of regions of the heart

5′ Impedance changes of regions of the heart

10 Device for processing EIT data

11, 21, 31, 41 Steps in sequence 1

30 EIT device

33 Electrode array

33′ Electrodes

34 Thorax

35 Patient

36 Electrode array on the thorax in a normal position

36′ Electrode array in a position close to the abdomen

37 Distance, vertical position deviation

40 Measured value acquisition and feed unit

44 Regions of the lungs

44′ Regions of the lungs, improved visualization

55 Position 55 in space of the heart

50 Data input unit

70 Control unit, computing and control unit, ?C

77 Memory

90 Data output unit

93, 93′ Heart region

95 Display device

97, 97′ Lung regions

98 Operational controls

99, 99′, 99″ Elements of the display device 95

100, 100′ START

300 Data set of EIT data

400 First data set

400′ First output signal

500 Second data set

500′ Second output signal

551 Signal path

600 Additional data set

600′ Control signal

800 Graphic representation

801a, 801b Position of the electrode array on the thorax

802a, 802b Symbolic representation, arrows

803 Output field

900 Visualization

904, 904′, 904″ Representations of EIT image

940, 940′, 940″ Image regions in the EIT image

999, 999′ STOP

1000 Return

What is claimed is:

1. A device for determining a position in space of a heart region in relation to regions of the lungs in a thorax of a patient, the device comprising:
a data input unit configured to receive data and provide an electrical impedance tomography (EIT) data set;
a computing and control unit configured to determine a first data set with data that indicates spatial and local distributions of impedance values and/or impedance changes of regions of the lungs in the thorax and configured to process the first data set and the EIT data set and based thereon to determine a first output signal, which indicates a current position in space of regions of the lungs in the thorax; and configured to process the EIT data set to determine a second data set with data that indicates spatial and local distributions of the impedance values and/or impedance changes of regions of the heart in the thorax and configured to process the second data set and the EIT data set and based thereon to determine a second output signal, which indicates a current position in space of a heart region in relation to regions of the lungs in the thorax; and
a data output unit configured to provide the first output signal and configured to provide the second output signal.

2. A device in accordance with claim 1, wherein the EIT data set has signals or data belonging to at least a set of a plurality of electrodes, which set of electrodes is arranged in a horizontal plane around the thorax.

3. A device in accordance with claim 1, wherein the EIT data set has signals or data of at least two sets of a plurality of electrodes, which two sets of electrodes are arranged parallel to one another at a defined distance.

4. A device in accordance with one of the above claims, wherein the computing and control unit is configured to determine a position of an electrode array on the thorax of the patient based on the first data set and the second data set.

5. A device in accordance with one of the above claims, wherein the computing and control unit is configured to continuously determine the second data set from EIT data, and wherein the computing and control unit is further configured to take into account the second data set during the data processing of the chronologically later EIT data.

6. A device in accordance with claim 5, wherein the computing and control unit is configured to mark, mask or blank out subsets in the EIT data set on the basis of the second data set.

7. A device in accordance with claim 6, wherein the computing and control unit is configured to copy the marked or masked subsets from the EIT data set into another data set.

8. A device in accordance with claim 6, wherein the computing and control unit is configured to copy the subsets that were not blanked out from the EIT data set into another data set.

9. A device in accordance with claim 5, wherein the computing and control unit is configured to also take into account the marked or masked subsets or the blanked-out subsets when calculating a global impedance curve and/or when calculating regional impedance curves based on the EIT data set.

10. A device in accordance with claim 5, wherein:
the computing and control unit is configured adapt a data processing and/or signal filtering based on the second data set; and
the computing and control unit adjusts the data processing and/or signal filtering based on frequency ranges of patient cardiac activity, which frequency ranges are determined from the second data set.

11. A device in accordance with claim 10, wherein the data input unit is configured to input information concerning the heart rate from external data sources and to make the input information available to the computing and control unit for adaptation of the data processing and/or signal filtering.

12. A device in accordance with claim 1, wherein the computing and control unit is configured in interaction with the data output unit to take the determined position of the heart region into account in providing a visualization of the EIT data.

13. A process for operating a device for determining a position in space of a heart region in relation to regions of the lungs in a thorax of a patient, the process comprising the steps of:
providing the device, wherein the device comprises: a data input unit configured to receive data and provide an electrical impedance tomography (EIT) data set; a computing and control unit configured to determine a first data set with data that indicates spatial and local distributions of impedance values and/or impedance changes of regions of the lungs in the thorax and configured to process the first data set and the EIT data set and based thereon to determine a first output signal, which indicates a current position in space of regions of the lungs in the thorax; and configured to process the EIT data set to determine a second data set with data that indicates spatial and local distributions of the impedance values and/or impedance changes of regions of the heart in the thorax and configured to process the second data set and the EIT data set and based thereon to determine a second output signal, which indicates a current position in space of a heart region in relation to regions of the lungs in the thorax; and a data output unit configured to provide the first output signal and configured to provide the second output signal;
receiving the EIT data set;
determining the first data set of spatial and local distributions of impedance values and/or impedance changes of regions of the lungs in the thorax; and
determining the second data set of spatial and local distributions of impedance values and/or impedance changes of regions of the heart in the thorax.

14. A process for determining a position in space of a heart region in relation to regions of the lungs in a thorax of a patient, the process comprising the steps of:
providing an electrical impedance tomography (EIT) data set;
determining a first data set with data that indicate spatial and local distributions of impedance values and/or impedance changes of regions of the lungs in the thorax on the basis of the data set of EIT data;
determining and providing a first output signal that indicates a current position in space of regions of the lungs in the thorax based on the EIT data set as well as based on the first data set;
determining a second data set with data that indicate spatial and local -distributions of the impedance values and/or impedance changes of regions of the heart in the thorax based on the EIT data set; and
determining and providing a second output signal, which indicates a current position in space of a heart region in relation to regions of the lungs in the thorax, based on the EIT data set as well as based on the second data set.

15. A process in accordance with claim 13, wherein the EIT data set has signals or data of at least a set of a plurality of electrodes which set is arranged around the thorax.

16. A process in accordance with claim 13, wherein the EIT data set has signals or data of at least two sets of a plurality of electrodes, which sets of electrodes are arranged spaced apart from one another and parallel to one another at a defined distance.
